(19) United States
(12) Patent Application Publication
(10) Pub. No.: US 2024/0181112 A1
(43) Pub. Date: Jun. 6, 2024

(54) Uvc sanitation module and relative modular sanitation assembly

(71) Applicant: O3ZY SA, Stabio (CH)

(72) Inventor: James Mossi, Stabio (CH)

(21) Appl. No.: 18/285,003

(22) PCT Filed: Mar. 30, 2022
(86) PCT No.: PCT/IB2022/052925

§ 371 Date: Sep. 29, 2023

(30) Foreign Application Priority Data
Mar. 30, 2021 (IT) 102021000007910

(51) Int. Cl. A61L 9/20

(52) U.S. Cl. CPC A61L 9/20; A61L 2209/111; A61L 2209/12; A61L 2209/14; A61L 2209/16

(57) ABSTRACT

An air sanitation module having a housing body and at least a UV source hit by an air flow to be sanitised, wherein said housing body is of elongated shape and has a housing cavity aligned according to a longitudinal axis, said housing cavity has an inlet end opening and an outlet end opening on the two opposite sides of said longitudinal axis, said UV source is in the shape of a bundle of parallel, elongated UVC radiating lamps, said bundle of lamps being fully housed longitudinally within said housing cavity, and wherein blowing means are further provided at one of said ends of the housing cavity for establishing an air flow longitudinally to said housing cavity, said housing cavity having a circular cross-section and an inner surface provided with a surface treatment with antioxidant properties and reflecting UVC radiation.

### TECHNICAL FIELD OF THE INVENTION

The present invention relates to a modular UV sanitation device.

### Background Art

As is known, the need to eliminate pathogens in various environments is strongly felt, especially in critical health environments or in pandemic conditions. It is therefore desirable to have a device in place for effectively sanitising workplaces or other environments, such as hospitals, rest homes, schools, bars, restaurants, waiting rooms, offices, other public premises, means of transport (buses, trains, ships, lifts, etc.) and other environments occupied, for more or less time, by several people, and in particular for effectively sanitising the air in an environment and for reducing the possible contamination of the air and surfaces caused by indirect contamination by micro-organisms.

Various agents can be used to achieve the sanitation effect, including ultraviolet radiation, which has a known germicidal efficacy against viruses and bacteria, including Sars-Cov 2.

Ultraviolet radiation consists of electromagnetic waves with a wavelength less than that of light visible to the human eye and greater than X-rays: in other words, the wavelength range of ultraviolet radiation is typically 400-100 nm.

It is however known that the most effective wavelength is the one around 254 nm (therefore in the UVC field between 280-100 nm), which however has harmful effects on the eye and the skin, when not suitably protected, at doses already lower than those necessary for disinfection.

It should also be considered that the germicidal activity of the UVC rays on the exposed surfaces is effective if it occurs with direct radiation and at a reduced distance, but rays have no appreciable utility if used at a distance and not radiated directly on the surfaces or materials to be sanitised.

The germicidal effect of UVC is therefore currently only demonstrated at a close distance from the surfaces/materials to be decontaminated and used in areas forbidden to people.

There are already several solutions on the market which use UVC lamps as a germicidal agent, but none so far has been particularly effective, because suitable measures for using UV rays in optimal conditions have not been provided.

KR101796291 and US2015/0359921 disclose some examples of air purification systems using UV lamps.

US2012/0283508 discloses an air purifying device employing an elongated housing within which one or more UV lamps are inserted. A longitudinal air flow is created in the housing by means of a fan. Pre-filters, complex filtering elements and post-filters are included at both the inlet and the outlet of the housing, which reduce not only the VOC component in the air flow, but also any possible particulate matter.

However, the applicant has been able to verify in the field that this type of equipment is not really effective, both because it does not have an optimised configuration for the UV lamps, and because apparently excessive filtration is not beneficial for the result intended to be achieved.

Therefore, the object of the present invention is to provide a sanitation device with UVC radiation, which is optimised to obtain a high direct and indirect germicidal efficacy and a complete control of the environments in which it is made to operate, in total safety even in the presence of people.

It is also desired to provide a sanitation device of air and indirectly of surfaces, which is simple to use, can be modularly adapted to different applications and automatically adapts to varying operating conditions and wear of its components.

### SUMMARY OF THE INVENTION

The above objects are achieved, according to the present invention, with a modular sanitising device having the features defined in the attached independent claims.

Further preferred features of such a solution are defined in the dependent claims.

In particular, according to a first aspect of the invention, an air sanitation module is provided comprising a housing body and at least a UV source hit by a flow of air to be sanitised, in which
said housing body is of elongated shape and has a housing cavity aligned according to a longitudinal axis,
said housing cavity has an inlet end opening and an outlet end opening on the two opposite sides of said longitudinal axis,
said UV source is in the shape of a bundle of parallel, elongated lamps, emitting UVC radiation, said bundle of lamps being fully housed longitudinally within said housing cavity, and in which
blowing means are further included at one of said ends of the housing cavity for establishing an air flow longitudinally to said housing cavity,
and moreover
the housing cavity has a circular section and has an inner surface provided with a surface treatment with antioxidant and UVC radiation reflection properties,
the lamps of the bundle of lamps are equidistant so as to maintain a mutual maximum effective distance between a longitudinal axis of said lamps and between said longitudinal axis of the lamps to said inner surface of the housing cavity, and
the inlet and outlet end openings are free of filtering elements or are provided with a filtering element suitable to let particulate matter of size less than 100 μm pass through.

According to another aspect, the housing cavity has a surface roughness Ra less than about 0.9 μm, preferably less than 0.6 μm, and
a surface treatment of the housing cavity is provided, which is antioxidant and reflective for UVC frequencies (chemical nickel plating or PVD).

According to a preferred aspect, the blowing means are provided with a control for adjusting the operating speed determined as a function of a signal from an anemometric sensor, and power detection sensors (radiation level) of the UVC source are further included.

According to another aspect, the blowing unit is driven by said speed adjustment control so as to determine an air flow speed within the housing cavity which is a function of the length of said lamps of the bundle of lamps and of a lamp efficiency signal coming from said power detection sensors (radiation level).

Furthermore, preferably, the blowing unit is driven so that the “dose” of UVC delivered is from 30 to 38 mJ/cm², depending on the anemometric sensor signal and a signal from said power detection sensors (radiation level).

Preferably, the control unit is arranged to determine, based on a signal proportional to an energy absorption (current, voltage, . . . ) of said blowing unit and on a speed signal of said anemometric sensor, a saturation level of said filtering element, when included.

For example, said control for adjusting the speed of the blowing unit is a tachometric dynamo, an encoder or a resolver.

According to a preferred variant, the cylindrical housing cavity has a diameter of about 125 mm and said bundle of lamps is about 530 mm long.

Optionally, a shielding grid is included at said outlet end opening of the cylindrical housing cavity, to prevent said UV radiation from escaping.

The invention also relates to a sanitation assembly comprising a plurality of modules as described above, arranged adjacent to each other with the relative parallel longitudinal axes.

The assembly preferably comprises at least two modules and it is installed integral with a conduit of or directly inside an ATU (Air Treatment Unit) or CMV (Controlled Mechanical Ventilation).

### BRIEF DESCRIPTION OF THE DRAWINGS

Further features and advantages of the solution according to the present invention will anyhow be more evident from the following detailed description of a preferred embodiment thereof, provided merely by way of non-limiting example and illustrated in the accompanying drawings, in which:

FIGS. 1A, 1B and 1C are respectively side elevation, rear elevation and perspective views of a single module sanitation device according to a first embodiment of the invention;

FIGS. 2A, 2B and 2C are respectively side elevation, rear elevation and perspective views of an exemplary four-module device according to the first embodiment of the invention;

FIGS. 3A, 3B and 3C are respectively side elevation, rear elevation and perspective views of an exemplary twenty-module device according to the first embodiment of the invention;

FIG. 4 is a perspective view of an alternative embodiment to that of FIG. 3C, with a single blowing device;

FIG. 5 is a perspective view of a further alternative embodiment to that of FIG. 3C, without fans or filters, which is applied in an ATU or CMV conduit;

FIG. 6 is a perspective view of an alternative embodiment to that of FIG. 3C, with a single filtering element;

FIG. 7A is a perspective exploded view of a sanitation device according to another embodiment of the invention; and

FIG. 7B is a longitudinal sectional view of the device of FIG. 7A.

### DETAILED DESCRIPTION OF THE PREFERRED EMBODIMENT

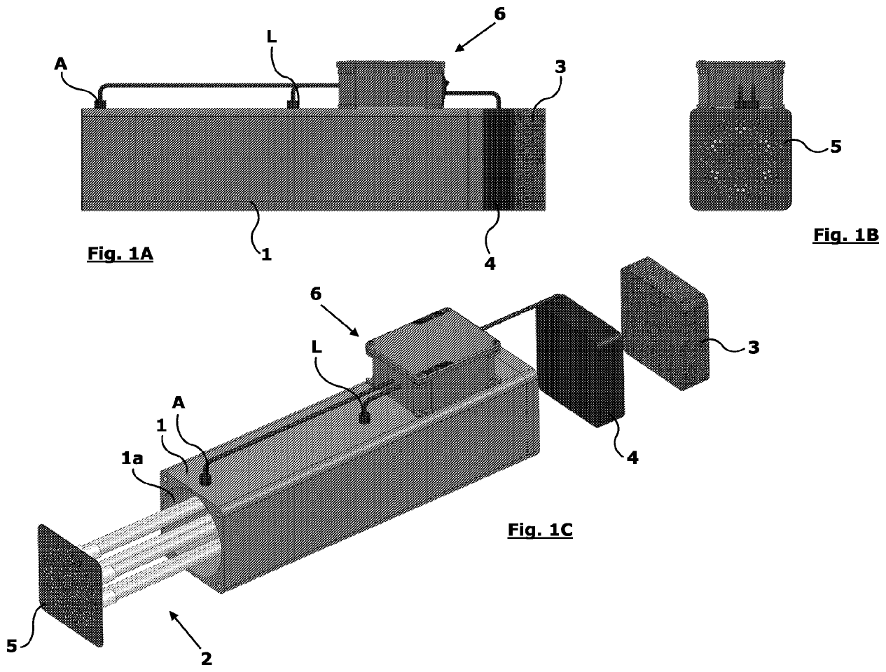

An exemplary single-module device according to the invention is shown in FIGS. 1A-1C.

An elongated housing body 1 has a housing cavity 1a in which a plurality of ultraviolet radiation lamps 2 are placed, in particular with UVC rays.

The housing body 1 is open at both ends, so that the housing cavity 1a also defines a flow conduit, within which an air flow can flow entering from a front inlet end and exiting from a rear outlet end. At the front end a filtering element 3 and blowing means 4 are arranged for dynamically pushing an air flow inside the cavity 1a and making it exit from the opposite end after lapping the lamps 2. A shielding grid 5 is preferably included at the rear outlet end, which has the purpose of adjusting the outgoing air flow and shielding the exit of ultraviolet light.

A control unit (not shown) is further included, in which a control logic, power supply units of the lamps 2 and of blowing means 4 and sensor receiving means of the device (which will be illustrated below) are incorporated. The control unit can be enclosed in a respective box 6, on which are located any switches or user control buttons. According to a variant, the control unit is installed remotely and the box 6 contains the wiring of the above-mentioned units and receiving means, together with a wireless transceiver which communicates with a digital communication protocol (Wi-Fi, for example) towards the remote control unit.

In the embodiment of FIG. 1C, there is a control unit in the respective box 6 for the single housing body 1 which defines a single modular element. In other embodiments (which will be illustrated below), a plurality of modular elements, each corresponding to a housing body, is served by a single wiring collection box and by a single control unit.

In the case of a commercial-type process control unit (like Raspberry™ or Arduino™ or others), the box 6 includes one or more interface boards for the connection of sensors, UVC lamps and fan and related circuit protection devices and anything else useful for the proper operation of the device. In the case of an embedded control unit, a solution is for example included in which the futures described above are integrated in the same boards, but solutions with multiple boards are not excluded.

A detailed description of the individual components is now provided, which is to be understood by way of non-limiting example, except for the essential aspects identified in the claims.

The housing body 1 is preferably formed by an aluminium extrusion with a cylindrical inner cavity, for example 125 mm in diameter. The length is ideally as high as possible, compatible with the overall dimensions of the device (for example if it must be transportable, or installable in a pre-existing system) and above all compatible with the existing standard lamps (in order to maintain industrially sustainable costs). For example, a preferred length is between 15 cm and 120 cm which, together with the 125 mm diameter, has been shown to be a good compromise between (i) air flow rate, (ii) number of lamps mounted inside at the correct distance to obtain the best efficiency, (iii) use of widespread commercial fans, and (iv) modularity in relation to the different solutions described below.

An important technical aspect is the inner surface state of the housing cavity 1a.

Following numerous performance tests, it has been noted that the surface of the cavity 1a must have a surface roughness Ra of at most about 0.9 μm and preferably less than 0.6 μm, and it is also important to arrange a surface treatment (mechanical treatment or coating) which simultaneously gives the material antioxidant properties and makes it adapted to reflect the frequency of the ultraviolet rays. For example, it has been identified that a chemical nickel plating treatment according to UNI ISO 4527 applied to the aluminium alloy of cavity 1a achieves the best compromise between reflectance features and treatment economy. Alternatively, it has been found that a PVD treatment, for example with aluminium oxide, is equally effective. However, there are also other effective treatments, but the greatest effectiveness has been demonstrated with a metal coating adapted to effectively reflect UVCs and at the same time offering good antioxidant properties of the surface.

With these conditions, the inner surface is advantageously capable of reflecting the ultraviolet emission radiated by the UVC lamps to the inner cavity 1a, so as to increase the radiation intensity being present in the cavity 1a and therefore reach a dose of UVC high enough to not require further treatments by photocatalysis.

The blowing means 4 are typically in the form of a standard fan (consisting of an electric motor and related impeller), of a diameter suitable for the inner diameter of the cavity 1a, advantageously provided with a tachometric dynamo for adjusting the rate of revolutions. The fan is electrically connected to the control unit in order to receive power supply and adjustment signals.

Due to the tachometric dynamo, it is possible to vary the flow rate and the speed of the air pushed and/or drawn by the fan 4 according to the signals coming from the control unit. The tachometric dynamo also performs a diagnostic function, since a feedback signal of the tachometric dynamo to the control unit provides values which can be used to detect the operation of the fan itself.

The fan 4 draws the air from the environment, through the inlet opening of the cavity 1a, possibly provided with the filter 3, to make it flow longitudinally inside the elongated cavity 1a, until it exits from the grid 5.

The device also has an anemometric sensor A (described below) which detects the air speed inside the cavity 1a and provides data related to the air speed which are used to control the rotation of the fan. The speed adjustment of the fan 4 is important in order to determine the flow rate and speed of the air inside the cavity 1a, so that the air passing inside the cavity 1a remains in close proximity to the UVC sources (the lamps 2) for a predetermined time, necessary for the total sanitation of the air itself. The speed parameter—which is detected by the anemometric sensor A—obviously depends on the length of the lamps 2 and their irradiation power, because the parameter to be respected is an average air residence time near a certain energy of the UVC source.

For example, UVC lamps with the current technology are about 430 mm long and with a diameter of 125 mm allow to work with a flow rate of about 150 m³/h, and an air speed of about 3 m/sec.

The basic parameters to be combined to obtain good sterilisation are the residence time of the air near the UVC source, the maximum distance of the air flowing from the UVC source (which will be further discussed below) and the power of the UVC sources. These parameters, suitably combined, determine the “dose” of UVC and consequently the sterilisation level of the treated air. In fact, typically in order to be able to scientifically certify the sanitation of the air flow, air must be exposed to a certain radiation energy (mJ/cm²) which—with the device according to the invention—can be achieved by knowing the radiation power of the lamps 2 and the air residence time near the lamps 2 (in turn derivable from the anemometric measurement and the geometric length of the flow path within the housing body).

The filtering element 3, if included, consists of a coarse filter, suitable for filtering dirt of large dimensions suspended in the air (for example feathers, insects, hair, . . . ), which could compromise the operation of the fan and/or of the lamps.

According to an essential feature of the invention, the filtering element must let particulate elements of dimensions less than 100 μm, such as typically airborne dust, pass through. In fact, it has been noted that, surprisingly, maintaining the filtering power of the filters 3 within these limits greatly improves the sanitation effectiveness of the device. It is believed that this depends on the fact that dust is an important carrier for pathogens (e.g., viruses and bacteria) and therefore it is advantageous for it to be passed through the cavity 1a instead of blocking it in or upstream of the filter 3.

Moreover, the particles of dust which are sanitised inside the cavity 1a, exit the grid 5 and settle on the surfaces, thus contributing to the sanitation of said surfaces (in addition to the circulating air).

When the filter becomes saturated with the dirt suspended in the air, it causes an increase in the prevalence and a consequent reduction in the flow rate/speed of the entering air, which is compensated by the control unit by an increase in the power supply which accelerates the rotation speed of the fan 4. Therefore, the fan current absorption measurement, combined with the anemometer reading signal, allows the saturation level of the filtering element 3 to be determined automatically. In fact, with the same air speed detected by the anemometer, the greater the amount of current absorbed and the greater is the saturation level of the filter: beyond a predetermined current absorption threshold, the control unit establishes that the filter 3 is to be replaced or regenerated and sends a warning signal to a warning unit, for example a sound or light emitter or a software component (for example an app on a smartphone) which makes the need to replace the filter clear to the user. In the case of application in ATUs or CMVs (as will be seen below) it is possible to signal a warning directly on the ATU or CMV control panel.

Although embodiments have been illustrated in the figures in which the filtering element 3, where included, is arranged only at the upstream end of the housing body, the filtering elements depicted can be positioned either at the upstream end, or at the downstream end or in both positions. When the filtering element 3 is arranged at the downstream outlet end, it is possible to included active-carbon elements for removing organic molecules (VOCs) or other gaseous pollutants which can be downstream of the UV treatment.

The ultraviolet source is defined by a plurality of elongated UVC lamps, for example cylindrical lamps (which however are not to be understood as limiting), arranged longitudinally parallel in a bundle inside the cavity 1a. The bundle of lamps 2 forms an ultraviolet source arranged to emit ultraviolet rays at a defined radiation. The ultraviolet source can comprise one or more gas discharge, mercury vapour lamps, one or more LED (Light Emitting Diode) sources or other current or future technologies, having the same purpose.

As already mentioned above, the ultraviolet source is arranged to emit ultraviolet radiation with a wavelength between 100-300 nm, preferably an ultraviolet source is used at 254 nm: this wavelength has a particular germicidal effect which exploits the breakdown of nucleic acids, RNA and DNA, of micro-organisms. The device thus equipped can be adapted to radiate with ultraviolet radiation the air drawn from the environment with an energy, for example, between 10-50 mJ/cm². In this regard, it is to be noted that at about 30-38 mJ/cm² it is possible to inactivate 99.99% of pathogens.

As noted above, the germicidal activity of ultraviolet rays on exposed surfaces is effective only by direct radiation, at the appropriate power and at a reduced distance from the source of the flowing air. In order to achieve maximum efficiency, it is therefore essential how the installation of the lamps inside the cavity 1a is made.

To this end, the lamps have an elongated shape, for example lengths between 125 and 1100 mm and a diameter of 15-20 mm, and are arranged longitudinally in a bundle in the cavity 1a. The air flows in the direction of the length of the lamp, along the longitudinal axis of the housing cavity 1a.

The mutual positioning between the individual lamps of the bundle and with respect to the inner wall of the cavity 1a is also relevant. In the case of the first embodiment, shown in FIGS. 1A-6, six lamps at 60° to each other are positioned inside the extruded body and spaced by a diameter adapted to maintain a space between the lamps and from the lamps to the inner surface of the cavity 1a, less than an effective distance of the UVC sources. With the UVC sources currently available in the industry, the effective distance is around 30 mm.

With this construction, the air flowing in the cavity 1a never passes at a distance greater than the effective distance (for example 30 mm) from the UVC irradiation source and the maximum germicidal activity is obtained.

In the case of a housing body 1 with very different sizes, the number and arrangement of the lamps can also change, so as to achieve air flow paths which do not deviate by a distance greater than the effective distance from the UVC sources.

The device according to the invention also has a series of sensors which detect parameters useful for controlling operation.

As mentioned above, a lamp efficiency sensor L is included, i.e., a UV sensor (a photodiode provided with an electronic interface board), for example calibrated at 254 nm and adjusted with a maximum threshold at 1,000 mW/cm², which has the property of detecting the efficiency of the ultraviolet source and emitting a signal (for example, in the case of an analog sensor, a variable voltage 0-5 V) proportional to the radiation intensity.

This is an important information—which is made available to the user by means of a warning device, for example a section of a software application which runs on a smartphone—because, in order to ensure germicidal effectiveness, the ultraviolet source must have constant operating standards; thereby, the replacement of the lamps does not occur according to use (estimated life hours by the lamp manufacturer) but as a result of a certain measurement which leads to exceeding a lower efficiency threshold. Furthermore, the detection of the actual power of the UV source (for example measured in mW/cm²), as seen above, is useful information to be able to establish with certainty—even as the UV source loses efficiency with use—the amount of energy per unit area delivered to the air flow and therefore be able to certify the actual sanitation of the air.

Another important sensor, already mentioned above, is an anemometer or flow meter A. This anemometric sensor A has the purpose of measuring the actual air speed near the rear or outlet end of the housing body 1, near the grid 5. In this case, for example, a 4-20 mA digital anemometer can be used. This anemometric sensor A performs several functions:

1. The air speed reading influences the adjustment of the fan 4, to determine the residence time of the air in contact with the ultraviolet source. The control unit, according to the detected speed value of the anemometer A, is able to adapt the rotation speed of the fan 4 (since it is provided with a specific tachometric dynamo for this purpose) and therefore return the air speed level to the desired parameters regardless of the filter conditions 3.

2. The air speed reading, combined with the rotation speed of the fan 4, allows to establish the saturation level of the filter 3: as the anemometer signal imposes a higher rotation speed of the fan 4, a progressive saturation of the filter is detected, until a threshold speed of the fan 4 is reached which triggers the output of an alarm signal.

3. The air speed reading, combined with the technical features of the components, achieves a diagnostic function; for example, if the control unit sets a certain rotation speed of the fan 4 to which an estimated air speed should correspond, but said estimated speed is not detected by the anemometer A at values exceeding the possible saturation of the filter, it may mean that the fan 4 is faulty.

Finally, a temperature sensor and a humidity sensor (not shown) are preferably also included for the measurement of the temperature of the housing body 1 and of the exiting air and humidity. The temperature and humidity detection can have a diagnostic and safety function. In case of application in ATUs or CMVs, said sensor measurements can be used as a feedback signal for checking the temperature of the air in transit.

As can be seen from the above description, the device according to the invention can be configured as a very efficient, compact sanitation module, arranged so as to be easily coupled to similar modules. The elongated parallelepiped shape of the housing body 1, with inlet/front and outlet/rear ends aligned according to the longitudinal axis is well suited for incorporation into a more complex apparatus and suitable for intercepting a greater air flow rate. Thereby, it is possible to obtain operating assemblies suitable for high flow rates of highly sanitised air, intended for environments of significant dimensions and above all integrable in ATUs (Air Treatment Units) and CMV (Controlled Mechanical Ventilation), so as to contribute to the sanitation of conduits in pre-existing air conditioning systems (theoretically mandatory sanitation that is rarely performed due to complexity and costs). Obviously, said assemblies lend themselves to being integrated into newly built ATU and CMV systems but also as stand-alone units.

The complex apparatus, consisting of an assembly of individual operating modules, can thus represent a reliable aid which can also be used in hospitals and healthcare facilities in general.

The apparatus is modular, in the sense that the resulting assembly can be assembled of an indefinite number of units, assembled together in a composition preferably but not necessarily with a rectangular cross section.

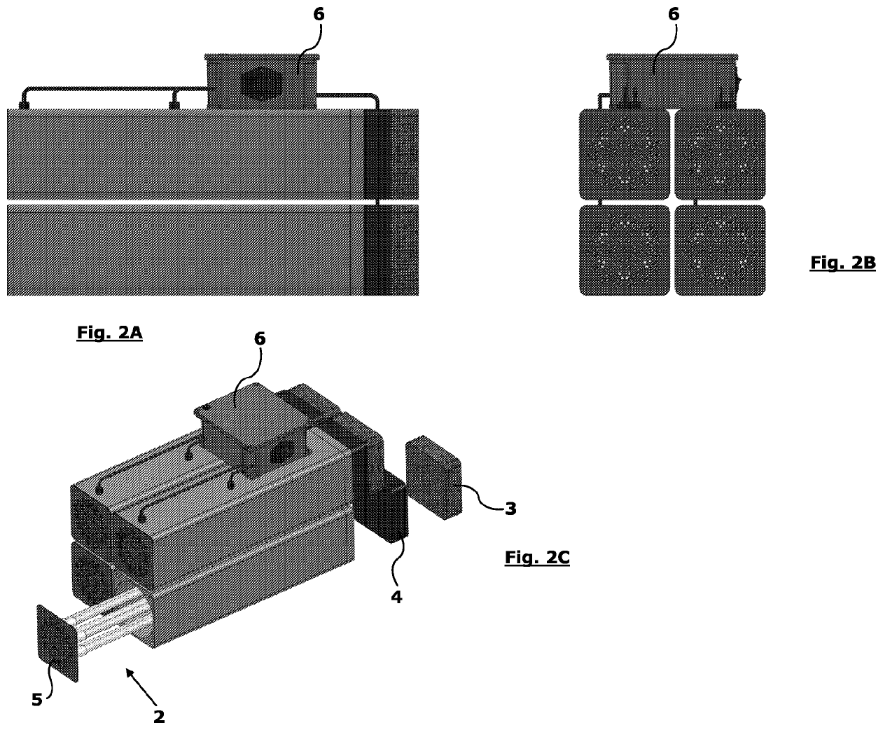

An assembled assembly with four modules such as those of FIG. 1C is depicted in FIGS. 2A-2C. The four modules are consolidated together, for example by means of special retaining brackets (not shown) of the four housing bodies arranged parallel to each other and coupled laterally two by two. Only one box 6 for the wiring and any control unit is associated with the assembly of four modules.

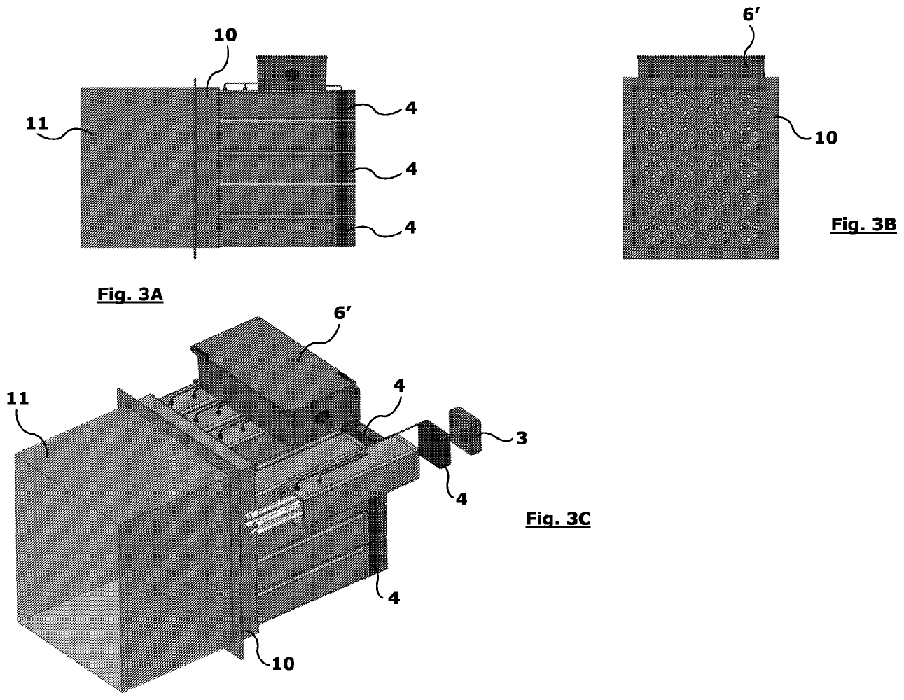

FIGS. 3A-3C show a different embodiment, with an assembly consisting of 4×5 units adjacent to each other in parallel (for purposes of clarity only, a unit is drawn away from the group and exploded). Also in this case, there is only one box 6′ for connecting the wiring. The modules are kept coupled together and are joined to an overall frame 10 which defines a single outlet port, to which one end of a delivery conduit 11 is fixed. In this case it is preferable to do without the outlet grids of each module, since the light of the UVC sources is still shielded by the presence of the conduit 11. The modular assembly is an independent unit which can be used standalone in large environments. The blowing units 4 can work both pushing and sucking with respect to the UVC sources.

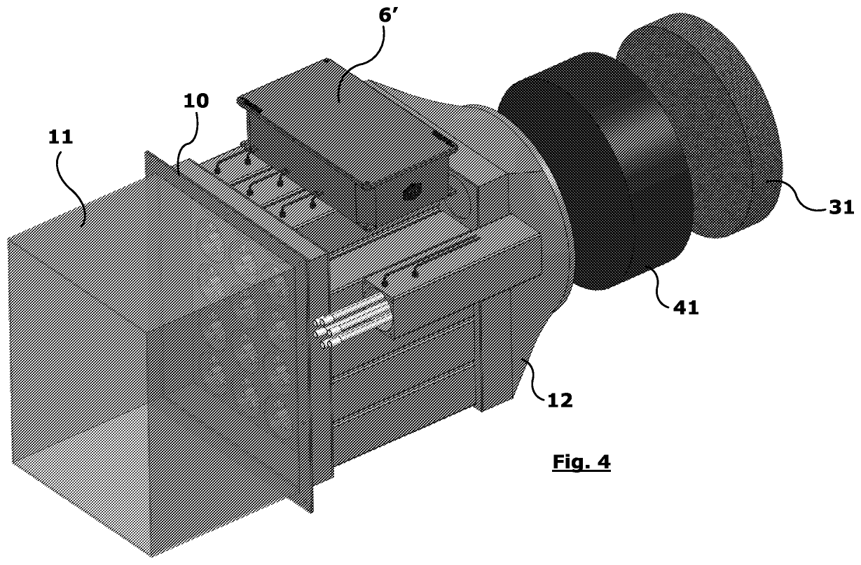

FIG. 4 shows another embodiment similar to that of FIG. 3C, with an assembled assembly with 4×5 modules according to the invention. In this case, a single blowing unit 41 is included, of large capacity, provided with a respective single filter 31. To connect the blowing unit 41, for example also of circular shape, with the inlet ends of the modules, a diverter conduit 12 is included: the air flow which passes through the blowing unit 41 is therefore distributed by means of the diverter conduit towards all the inlet ends of the housing cavities of the twenty modules. Also in this case there is a single wiring connection box 6′ for all the modules. The blowing unit 41 can work either pushing air towards or sucking air from the UVC sources. The filter 31 can be mounted upstream of the unit 41 with respect to the air flow or at the downstream end or at both ends.

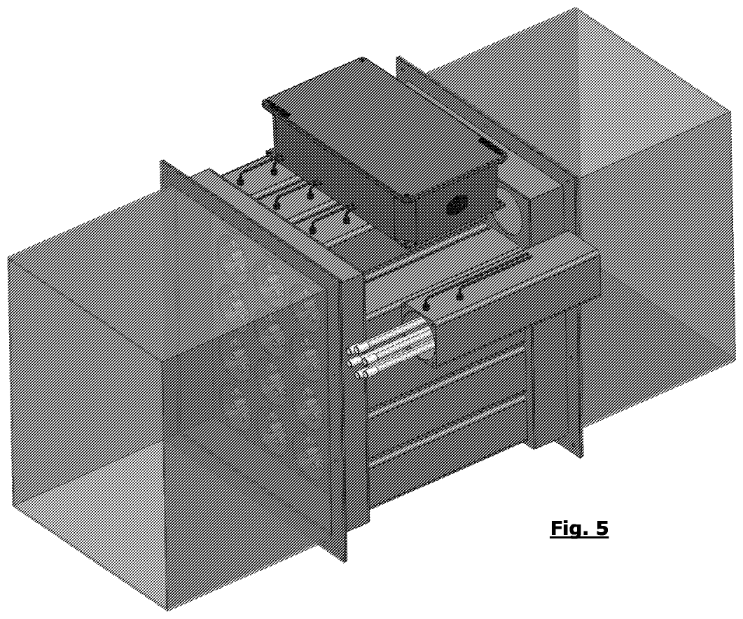

FIG. 5 shows yet another embodiment of a sanitation assembly similar to that of FIG. 3C. In this case, the assembly is configured to be integrated into an ATU or CMV conduit. In the drawing of FIG. 5 an assembly of 4×5 modules is shown which, with the example dimensions indicated above for the individual modules, is adapted to be integrated in a conduit with a section of about 40×70 cm, typical of an ATU with a flow rate of 6000 m³/h and an air speed of 6 m/sec (standard configuration used in industrial and commercial environments but also in hospital facilities). In this case there are no blowing units or filters, since these components are integral parts of the ATU or CMV systems.

In these versions, in which the sanitation assembly is integrated in structures and aeration systems (such as ATUs or CMVs), it is envisaged to install an actual electrical power panel (not shown), where the installed powers require it for compliance with current regulations, with integrated control unit and with the arrangement for a Wi-Fi interface or with a cable connection for connection to the control panels of the external units (ATUs, CMVs, etc.). This electrical panel consists mainly, but not limited to, a power section for controlling the lamps and possibly the blowing units (where included). There is also a low voltage section (e.g., 24 Vcc, but possibly 12 Vcc and 5 Vcc) for the process control unit in addition to everything necessary for the connection of the components described above and for a Wi-Fi connection according to the current technical standards or for a cable connection.

In the case of a commercial-type process control unit (such as Raspberry™ or Arduino™ or others), as already mentioned, one or more interface boards are included for the connection of the sensors, the UVC lamps and the blowing units and the related circuit protection devices and anything else useful for the proper operation of the assembly.

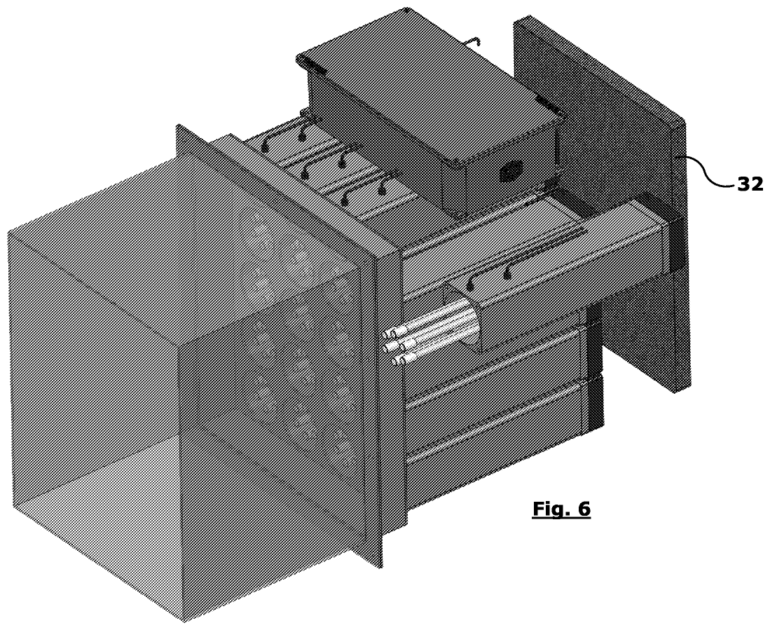

Another variant, similar to that of FIG. 30, is shown in FIG. 6. In this case, the individual filters 3 dedicated to each module are replaced by a single filter 32 with dimensions such as to be arranged in front of the inlet ends of all the modules. This group is also conceived to be an independent apparatus, usable stand-alone in large environments. It should be noted that the blowing units can work either pushing air towards or sucking air from the UVC sources.

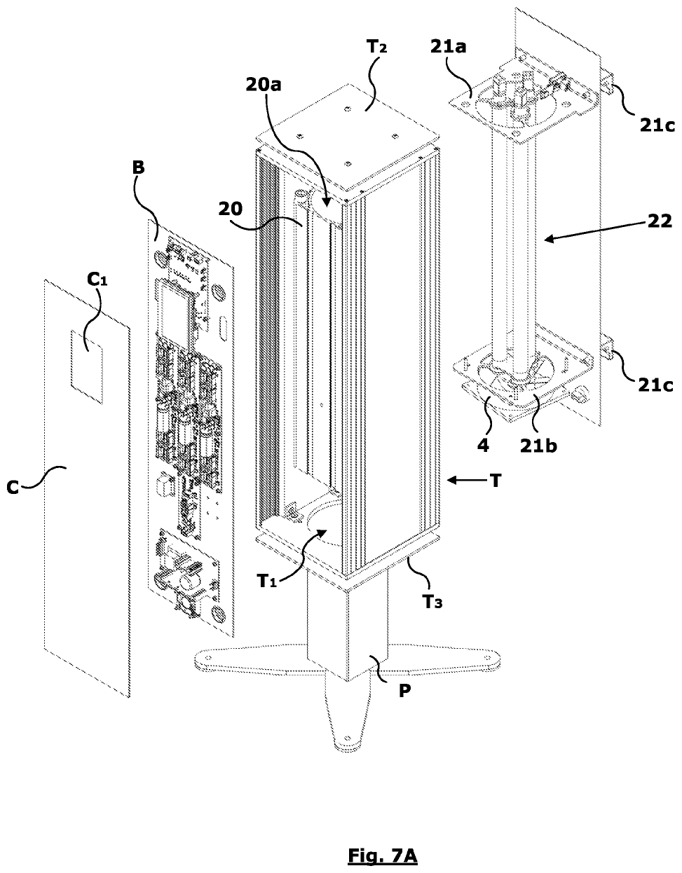

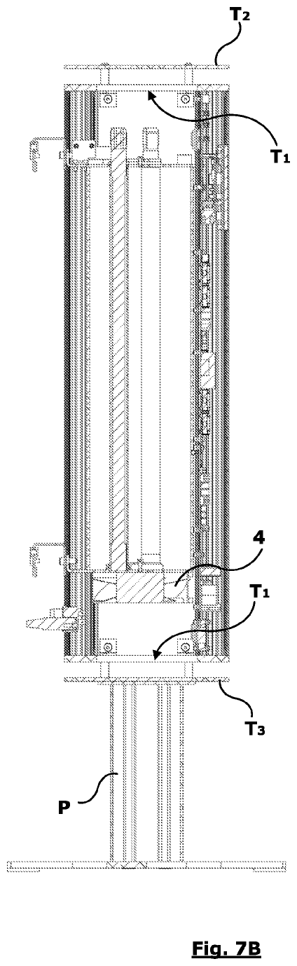

FIGS. 7A and 7B show a further embodiment of a stand-alone apparatus provided with a module according to the invention.

In this case, a module according to the invention is included, with a cavity 20a which houses three UVC lamps 22. The three lamps 22 are kept at the correct distance from each other, at 120° from each other, by means of a pair of end holding frames 21a and 21b. The frames 21a and 21b are attached to the edge of the inlet and outlet openings of the housing body 20.

A fan 4 is preferably mounted on one of the two frames 21b.

The sanitisation module is enclosed within a casing T having a pleasant appearance, for example supported autonomously at the ground by means of a pedestal P. The casing has inlet and outlet openings of the air T⋅, without any filters but simply shielded by plates T₂ and T₃ mounted briefly spaced from the openings so as to prevent dirt to enter.

The control unit is arranged on a printed circuit board B which is integrated with a side of the casing T. Preferably the printed circuit board is covered by a flat cover C provided with a cut-out C₁ through which a small touch screen or other I/O device belonging to the printed circuit board B is visible.

The sanitisation module, without the pedestal P, can be mounted on the wall in a horizontal or vertical position, or on the ceiling, exploiting appropriate brackets 21c.

As can be seen from the above description, the device and the module according to the invention perfectly satisfy the objects set forth in the premise. In fact, the specific configuration of the module allows to obtain a high efficiency of exploitation of UVC radiation for sanitising the air. Furthermore, the same configuration makes it easy to assemble a plurality of modules in a sanitising unit which can be used both as a stand-alone unit and integrated into conduits of industrial ventilation systems or in sanitary environments.

It should be noted that, advantageously, the anemometric sensor and the UV radiation sensor are useful, on the one hand, to certify the germicidal effectiveness of the device based on scientifically proven effects, and on the other, to provide feedback to the control unit, to adjust the operation of the blowing means and to trigger a prompt for replacement of the filter (if present) when it is excessively saturated and of the lamps at the end of their useful life.

It is understood that the invention is not to be considered as limited by the particular embodiments described and illustrated, but different variants are possible, all within the reach of a person skilled in the art, without departing from the scope of the invention itself, which is exclusively defined by the following claims.

In particular, the features illustrated in the individual embodiments can also be adopted in different embodiments, where compatible: for example, the single filter of FIG. 6 can also be included in the embodiment of FIG. 2C and so on.

1. Air sanitation module comprising a housing body (1) and at least a UV source (2) hit by an air flow to be sanitised, wherein
said housing body (1) is of elongated shape and has a housing cavity (1a) aligned according to a longitudinal axis,
said housing cavity (1a) has an inlet end opening and an outlet end opening on the two opposite sides of said longitudinal axis,
said UV source is in the shape of a bundle of parallel, elongated UVC radiating lamps (2), said bundle of lamps (2) being fully housed longitudinally within said housing cavity (1), and wherein
blowing means (4) are further included at one of said ends of the housing cavity (1a) for establishing an air flow longitudinally to said housing cavity (1a),
characterised in that
said housing cavity (1) has a circular cross section and has an inner surface provided with a surface treatment having antioxidant properties and reflecting UVC radiation, and
said lamps of the bundle of lamps (2) are equally spaced apart so as to maintain a mutual maximum effective distance between a longitudinal axis of said lamps and between said longitudinal axis of the lamps to said inner surface of the housing cavity (1a), and in that
said inlet and outlet end openings are free of filtering elements or provided with a filtering element (3) suitable to let particulate matter of dimensions less than 100 μm pass through.

2. Air sanitation module according to claim 1, wherein the housing cavity (1a) has a surface roughness Ra less than about 0.9 μm, preferably less than 0.6 μm, and
said surface treatment of the housing cavity (1a) is a chemical nickel plating or PVD.

3. Air sanitation module according to claim 1, wherein said blowing means (4) are provided with a operating adjustment drive determined as a function of a signal from an anemometric sensor (A), and
power (radiation level) detection sensors (L) of said UV source are further included.

4. Air sanitation module according to claim 3, wherein said blowing unit (4) is driven by said speed adjustment drive so as to adjust an air flow speed within the housing cavity (1a) which is a function of the length of said lamps of the bundle of lamps (2) and of a lamp efficiency signal coming from said power (radiation level) detection sensors (L).

5. Air sanitation module according to claim 3, wherein, as a function of said anemometric sensor signal (A) and a signal from said power (radiation level) detection sensors (L), said blowing unit (4) is driven so that the “dose” of UVC delivered is from 30 to 38 mJ/cm².

6. Air sanitation module according to claim 1, wherein said control unit is arranged to determine, based on a signal proportional to an energy absorption (current, voltage, . . . ) of said blowing unit (4) and on a speed signal of said anemometric sensor (A), a saturation level of said filtering element (3).

7. Air sanitation module according to claim 1, wherein said speed adjustment drive of the blowing unit (4) is a tachometric dynamo, an encoder or a resolver.

8. Air sanitation module according to claim 1, wherein said cylindrical housing cavity (1a) has a diameter of about 125 mm and said bundle of lamps (2) is about 530 mm long.

9. Air sanitation module according to claim 1, wherein a shielding grid (5) is provided at said outlet end opening of the cylindrical housing cavity (1a), to prevent said UV radiation from escaping.

10. Air sanitation module according to claim 1, wherein said housing body (1, T) is an aluminium extruded body.

11. Sanitation assembly, characterised in that it comprises a plurality of modules according to claim 1, arranged adjacent to each other with the relative parallel longitudinal axes.

12. Assembly according to claim 11, wherein at least two modules are provided and it is installed integrated in a conduit of or directly inside an ATU (Air Treatment Unit) or CMV (Controlled Mechanical Ventilation).
